(19) United States
(12) Patent Application Publication
(10) Pub. No.: US 2026/0216208 A1
(43) Pub. Date: Jul. 30, 2026

(54) TREATMENT FOR AZIDE POISONING

(71) Applicant: The Regents of the University of California, Oakland, CA (US)

(72) Inventors: Gerard R. Boss, La Jolla, CA (US); John Tat, San Diego, CA (US); Adriano Chan, San Diego, CA (US)

(21) Appl. No.: 19/148,610

(22) PCT Filed: Jan. 26, 2024
(86) PCT No.: PCT/US2024/013063

§ 371 Date: Jul. 16, 2025

Related U.S. Application Data
(60) Provisional application No. 63/481,675, filed on Jan. 26, 2023.

(51) Int. Cl. A61K 31/555; A61K 9/00; A61P 9/02

(52) U.S. Cl. CPC A61K 31/555; A61K 9/0019; A61P 9/02

(57) ABSTRACT

Compositions and methods for treatment of azide poisoning in a subject in need thereof, having an effective amount of cobinamide, a cobinamide derivative, or a salt thereof. Azide poisoning can be caused for example by exposure to sodium azide.

### CROSS-REFERENCE TO RELATED APPLICATIONS

This application claims priority to U.S. Provisional Application No. 63/481,675, filed Jan. 26, 2023, the entire contents of which are incorporated by reference.

### GOVERNMENT SPONSORSHIP

This invention was made with government support under grant No. NS058030 and No. NS087964 awarded by the National Institutes of Health. The government has certain rights in the invention.

### TECHNICAL FIELD

The present invention relates to a treatment for azide poisoning.

### BACKGROUND

Sodium azide (NaN₃) is used in a variety of industries with over 1,000 tons produced annually¹. It is highly toxic, with the human lethal dose estimated to be ~700 mg or ~10 mg/kg². Fortunately, azide poisoning is rare with 156 reported cases worldwide between the years 2000 and 20203. However, this infrequency may prove detrimental in a mass casualty event, such as an industrial accident or terrorist attack—most medical personnel will not have encountered an azide-poisoned patient and therefore not be well-informed about azide toxicity and treatment options. A major terrorist attack is possible, since azide may be purchased through online retailers, and it has been used in several planned and executed attacks⁴⁻⁹. On several occasions, azide was used to poison communal beverages, leading to high casualties and highlighting azide's potential as a terrorist weapon¹⁰⁻¹⁴. Moreover, azide is a common suicidal agent, especially among laboratory workers, likely due to its common presence in research laboratories^{2,3,15}.

Symptoms occur within minutes of azide exposure, ranging from dizziness, nausea, vomiting, and restlessness at low doses, to seizures, hypotension, metabolic acidosis, coma, and respiratory failure at high doses. Like carbon monoxide, cyanide, hydrogen sulfide, and methyl mercaptan, azide inhibits cytochrome c oxidase in complex IV of the mitochondrial electron transport chain, thereby reducing ATP production¹⁶⁻¹⁹. This results in a compensatory increase in glycolysis with attendant metabolic acidosis. Inhibiting complex IV increases mitochondrial electron leakage, thereby increasing superoxide anion (O₂·⁻) generation. Consistent with this mechanism, brain, lung, and heart tissues taken from azide-poisoned rats showed significant increases in malondialdehyde concentrations, a marker of lipid peroxidation²⁰. Through a poorly understood mechanism, azide appears to be converted to nitric oxide (NO)²¹⁻²³, which likely underlies the profound hypotension that can occur in azide-poisoned victims.

No specific antidote is available for azide poisoning and treatment is solely supportive³. Due to mechanistic similarities between azide and cyanide, azide poisoning victims have been given cyanide antidotes such as sodium thiosulfate, amyl nitrite, sodium nitrite, or hydroxocobalamin (referred to subsequently as “cobalamin”)^{3,12,24-26}. Sodium thiosulfate detoxifies cyanide by converting it to thiocyanate, but no evidence exists that sodium thiosulfate reacts with azide. Amyl nitrite and sodium nitrite generate methemoglobin, which has a high affinity for cyanide, but binds azide only weakly²⁷⁻²⁸; nitrite might exacerbate azide-induced hypotension by virtue of its reduction to nitric oxide. Cobalamin is a rational treatment for azide poisoning because it binds both azide and NO, and thereby could potentially neutralize both species²⁹⁻³¹. However, cobalamin has low water solubility, necessitating administration in relatively large volumes via intravenous infusion. It, therefore, does not lend itself well to pre-hospital use.

Cobinamide is the penultimate precursor in the biosynthesis of cobalamin, lacking a dimethylbenzimidazole ribonucleotide group coordinated to the lower axial position of the cobalt atom (FIGS. 1A-1B)³². Absence of the dimethylbenzimidazole ribonucleotide group imparts several chemical differences between cobinamide and cobalamin: (i) cobinamide has two, rather than one, ligand binding site; (ii) cobinamide has higher affinity for ligands³³; (iii) cobinamide is more easily oxidized and reduced³⁴; (iv) cobinamide is more water soluble, providing the potential for intramuscular administration in the field; and (v) cobinamide reacts more readily^{30,34-35} with NO and O₂·⁻.

### SUMMARY OF THE INVENTION

Disclosed herein are compositions and methods for treating azide poisoning.

In an aspect, a method of treating azide poisoning in a subject is provided, where the method includes administering to the subject an effective amount of cobinamide, a cobinamide derivative, or a salt thereof. In embodiments, the subject is a human, and the effective amount is from about 20 mg/kg to about 40 mg/kg, preferably about 30 mg/kg. In some embodiments, the cobinamide, cobinamide derivative, or salt thereof is administered orally. In other embodiments, the cobinamide, cobinamide derivative, or salt thereof is administered by intravenous injection. In embodiments, the azide poisoning is due to sodium azide exposure.

In embodiments, the azide poisoning is treated by increasing intracellular adenosine triphosphate (ATP) in azide-exposed cells. In embodiments, the azide poisoning is treated by inhibiting an increase in malondialdehyde in azide-exposed cells. In embodiments, the azide poisoning is treated by decreasing apoptosis in azide-exposed cells.

In another aspect, a pharmaceutical composition is provided, where the pharmaceutical composition includes an effective amount of cobinamide, a cobinamide derivative, or a salt thereof, and where the pharmaceutical composition is effective to treat azide poisoning in a human subject. In embodiments, the azide poisoning is due to sodium azide exposure. In embodiments, the effective amount is from about 20 mg/kg to about 40 mg/kg, preferably about 30 mg/kg. In some embodiments, the pharmaceutical composition is configured for oral administration. In other embodiments, the pharmaceutical composition is configured for intravenous administration.

In embodiments, the pharmaceutical composition is effective to increase intracellular adenosine triphosphate (ATP), inhibit an increase in malondialdehyde, and/or decrease apoptosis in azide-exposed cells. In embodiments, the pharmaceutical composition is effective to increase blood pressure and/or peripheral blood pressure in response to azide poisoning-induced vasoconstriction and/or hypotension.

### BRIEF DESCRIPTION OF THE DRAWINGS

FIGS. 1A-1B. Structures of hydroxocobalamin and aquohydroxocobinamide. Comparison between (A) hydroxocobalamin and (B) aquohydroxocobinamide at neutral pH. Cobinamide lacks the dimethylbenzimidazole ribonucleotide group coordinated to the lower axial position of the cobalt atom in hydroxocobalamin.

FIGS. 2A-2F. Cobinamide binds azide and rescues cells from azide toxicity. FIGS. 2A-2B. Sodium azide was added to a 25 μM aquohydroxocobinamide (A) or 25 μM hydroxocobalamin (B) solutions at final concentrations ranging from 0.25× to 16× of the cobinamide/cobalamin concentration. Ultra-violet-visible spectra were recorded from 300 to 700 nm. The insets are a plot of the change in the absorbance of cobinamide/cobalamin at 515 nm versus the NaN₃ concentration. FIGS. 2C-2D. A549 cells were treated with varying concentrations of sodium azide (50 μM to 100 mM) for 48 hours in the absence or presence of 25 μM aquohydroxocobinamide (C) or 25 μM hydroxocobalamin (D); the cells were then harvested and counted. Data are normalized to controls set at 100% and represent the mean z SEM of four independent experiments done in duplicate. The comparison of azide exposure alone versus azide plus aquohydroxocobinamide or hydroxocobalamin was analyzed by two-way ANOVA. FIG. 2E. A549 cells were seeded overnight at a low density, and then treated with the indicated drugs for 24 hours. The cells were washed with PBS, released in drug-free medium for 15 days, and then stained with 0.25% w/v crystal violet and counted. FIG. 2F. Quantification of the data in (E). Data are normalized to controls set at 100% and represent the mean±SEM of six independent experiments: statistical analysis was done by one-way ANOVA. *P≤0.05, **P≤0.01, and ***P<0.001. Cbi=cobinamide; NaN₃=sodium azide; IC₅₀=azide concentration where 50% inhibition occurs; Untr=untreated; Kₐ=binding affinity.

FIGS. 3A-3E. Mechanisms of azide toxicity; reversal by cobinamide. FIGS. 3A-3B. COS-7 cells (A) or A549 cells (B) were incubated in glucose-free medium containing 25 mM galactose for 24 hours. The next day, cells were exposed for 24 hours to 0.1 mM sodium azide with or without 25 μM aquohydroxocobinamide. The cells were extracted in situ and ATP was measured in the extracts. Data represent the mean±SEM of six (A) and five (B) independent experiments, respectively. FIG. 3C. A549 cells were incubated for 24 hours with 0.1 mM and 1 mM sodium azide, in the absence or presence of 25 μM aquohydroxocobinamide and the intracellular malondialdehyde (MDA) content was measured 24 hours later. Data represent the mean±SEM of five independent experiments and were analyzed by one-way ANOVA. FIGS. 3D-3E. A549 cells were incubated for 24 hours with 0.1 mM and 1 mM sodium azide, in the absence or presence of 25 μM aquohydroxocobinamide and induction of apoptosis based on caspase-3 cleavage was measured by immunofluorescence. FIG. 3D. Fluorescence microscopy images of cleaved caspase-3 positive cells (green); Hoeschst stain (blue) indicates the DNA of cells. Images were taken at 20× magnification and each scale bar represents 100 μm. FIG. 3E. Quantification of the data in (D): the data are the mean±SEM of three to four independent experiments. All experiments were analyzed by one-way ANOVA. *P≤0.05, **P<0.01, and ****P≤0.0001. Cbi=cobinamide; NaN₃=sodium azide; ns=non-significant.

FIGS. 4A-4C. Cobinamide protects animals from acute sodium azide poisoning. FIG. 4A. D. melanogaster were exposed to ~1500 ppm hydrazoic acid gas. After 2 minutes, when all the animals had fallen motionless, they were removed from the hydrazoic acid and monitored every 60 minutes for 4 hours and then every 24 hours until 72 hours post exposure; recovery was defined as the ability to walk or fly. FIG. 4B. D. melanogaster were placed in vials containing gauze that had been saturated with solutions of either 100 mM sodium azide or sodium phosphate as the only source of water. Survival, as determined by the ability to walk or fly, was determined every hour for 8 hours. In both studies, some of the flies had been fed food containing 400 μM aquohydroxocobinamide or 400 μM hydroxocobalamin for one week prior to the experiment. Data represent the mean±SEM of seven independent experiments with 15 flies per condition in each experiment. The cobinamide- and cobalamin-treated flies were compared to control flies by two-away ANOVA; asterisks and pound symbols are for comparison of cobinamide and cobalamin, respectively, to untreated flies. FIG. 4C. Mice of both sexes (8 males and 8 females) and variable age (4-15 months old) received an intraperitoneal injection of 30 mg/kg sodium azide. Two minutes later, the animals received an intramuscular injection of 350 mg/kg of histidylcobinamide, 450 mg/kg hydroxocobalamin, (or water). The difference between the cobinamide- and cobalamin-treated animals compared to water-treated animals was analyzed by a by log-rank sum test; * and #P<0.05, **P<0.01, and **** and ####P<0.0001. Cbi=cobinamide; OHCbl=cobalamin; NaN₃=sodium azide; Veh, vehicle.

FIGS. 5A-5C. Therapeutic effect of cobinamide on blood pressure and peripheral body temperature following azide exposure. FIG. 5A. Mice of both sexes received an intraperitoneal injection of 20 mg/kg sodium azide or water. At the indicated times, animals were euthanized by carbon dioxide asphyxiation, and blood was collected via cardiac puncture. Serum nitrite and nitrate concentrations were measured using a modified Griess-reagent method. Data at each time point represent the mean Z SEM of 6-8 mice. FIGS. 5B-5C. Male and female mice received an intraperitoneal injection of 20 mg/kg sodium azide at t=0 minutes followed by an intraperitoneal injection of 300 mg/kg histidylcobinamide (or water) 20 min later (t=20 minutes). FIG. 5B. Blood pressure was assessed via tail cuff plethysmography and the data represent the mean SEM of 8 mice. FIG. 5C. Tail temperature was assessed via a non-contact laser thermometer and the data represent the mean z SEM of 7 mice. The data were analyzed by two-way ANOVA; *P<0.05, **P<0.01, and ****P<0.0001. Cbi=cobinamide; Inj=injection; NaN₃=sodium azide.

FIGS. 6A-6B. Cobinamide but not cobalamin protects cells from azide toxicity. Cells were treated with varying concentrations of NaN₃ (50 μM to 100 mM) for 48 hours in the absence or presence of 25 μM aquohydroxocobinamide (Cbi) (A) or 25 μM hydroxocobalamin (OHCbl) (B); the cells were then harvested and counted. Data are normalized to controls set at 100% and represent the mean±SEM of four independent experiments done in duplicate. The comparison of azide exposure alone versus azide plus cobinamide or cobalamin was analyzed by two-way ANOVA. IC₅₀=azide concentration where 50% inhibition occurs.

FIGS. 7A-7B. Cobinamide has no effect on mean arterial blood pressure or tail temperature. Male and female mice received an intraperitoneal injection of water at t=0, followed by an intraperitoneal injection of either 300 mg/kg histidylcobinamide (Cbi) or water 20 minutes later (t=20 minutes). FIG. 7A, Blood pressure was assessed via tail cuff plethysmography and the data represent the mean±SEM of 7 mice. FIG. 7B. Tail temperature was assessed via a non-contact laser thermometer and the data represent the mean±SEM of 7 mice.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, Molecular Cloning: A Laboratory Manual, 2nd ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20th ed., (Lippincott, Williams & Wilkins 2003), and Remington, The Science and Practice of Pharmacy, 22th ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a pharmaceutical composition, and/or a method that “comprises” a list of elements (e.g., components, features, or steps) is not necessarily limited to only those elements (or components or steps), but may include other elements (or components or steps) not expressly listed or inherent to the pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B. i.e., A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or mammalian animal subject to be treated.

As used herein the term “pharmaceutical composition” refers to a pharmaceutically acceptable composition, wherein the composition comprises a pharmaceutically active agent, and in some embodiments, further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently, or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a State government or listed in the U.S. Pharmacopoeia, other generally recognized pharmacopoeia in addition to other formulations that are safe for use in subjects, such as animals, and more particularly in humans and/or non-human mammals.

As used herein the term “pharmaceutically acceptable carrier” refers to an excipient, diluent, preservative, solubilizer, emulsifier, adjuvant, and/or vehicle with which a pharmaceutically active compound, such as a cobinamide compound, is administered. Such carriers may be sterile liquids, such as water and oils, including those of petroleum, animal, vegetable, or synthetic origin, such as peanut oil, soybean oil, mineral oil, sesame oil and the like, polyethylene glycols, glycerine, propylene glycol, or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington. The Science and Practice of Pharmacy, 20th ed., (Lippincott, Williams & Wilkins 2003). Except insofar as any conventional medium or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse, or slow progression of the disease, or otherwise reduce pathological consequences of the disease. In any case, an effective amount may be given in single or divided doses.

In some embodiments a “therapeutically effective amount” refers to an amount of one or more compounds, such as but not limited to cobinamide, which is sufficient to treat or ameliorate, or in some manner reduce the symptoms associated with azide poisonings.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g., a symptom associated with the disease or condition being treated. As such, “treatment” also includes situations where the disease, disorder, or pathological condition, or at least symptoms associated therewith, are completely inhibited (e.g., prevented from happening) or stopped (e.g., terminated) such that the patient no longer suffers from the condition, or at least the symptoms that characterize the condition.

As used herein, and unless otherwise specified, the terms “prevent.” “preventing” and “prevention” refer to the prevention of the onset, recurrence, or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or to prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

The invention contemplates the use of cobinamide, salts thereof, and cobinamide derivatives. A cobalt atom of the cobinamide and cobinamide derivatives independently may be coordinated with no ligands or one or more ligands, for example, one ligand or two ligands. As used herein, the phrase “cobinamide derivative” refers to a biologically active derivative (e.g., analog) of cobinamide, such as a heterocyclic or heteropolycyclic compound that is (i) coordinated with a central cobalt atom, and (ii) substituted with two or more alkyl substituents (e.g., four to eight alkyl substituents) that include at least one polar functional group, such as an amide, an ester, an ether, carboxylic acid, etc. The heterocyclic or heteropolycyclic compound may include 4 heteroatoms, such as nitrogen, oxygen, etc. The one or more ligands may include any ligand that is capable of coordinating with a cobalt atom, such as an unsubstituted or substituted tetrazole (e.g., an amino-tetrazole, an acetyl-tetrazole, etc.), an unsubstituted or substituted imidazole (e.g., acetyl-imidazole), histidine, etc. An exemplary cobinamide derivative is trihistidylcobinamide. As used herein, the phrase “amino-tetrazole” refers to a tetrazole moiety substituted at any one or more positions with (i) an amino moiety and/or (ii) a C1-C3 alkyl comprising an amino moiety. As used herein, the phrase “acetyl-tetrazole” refers to a tetrazole moiety substituted at any one or more positions with (i) an acetyl moiety and/or (ii) a C1-C3 alkyl comprising an acetyl moiety. As used herein, the phrase “acetyl-imidazole” refers to an imidazole moiety that is substituted at any one or more positions with (i) an acetyl moiety and/or (ii) a C1-C3 alkyl comprising an acetyl moiety.

In some embodiments, the one or more cobinamide derivative include an amino-tetrazole-cobinamide, a di-(amino-tetrazole)-cobinamide, an acetyl-tetrazole-cobinamide, a di-(acetyl-tetrazole)-cobinamide, an acetyl-imidazole-cobinamide, a di-(acetyl-imidazole)-cobinamide, or a combination thereof. An example of an amino-tetrazole-cobinamide is 5-amino-tetrazole-cobinamide. An example of a di-(amino-tetrazole)-cobinamide is di-(5-amino-tetrazole)-cobinamide. An example of an acetyl-tetrazole-cobinamide is 5-acetyl-tetrazole-cobinamide. An example of a di-(acetyl-tetrazole)-cobinamide is di-(5-acetyl-tetrazole)-cobinamide. An example of an acetyl-imidazole-cobinamide is 4-acetyl-imidazole-cobinamide. An example of a di-(acetyl-imidazole)-cobinamide is di-(4-acetyl-imidazole)-cobinamide.

The term “pharmaceutically active” as used herein refers to the beneficial biological activity of a substance on living matter and, in particular, on cells and tissues of the human body. A “pharmaceutically active agent” or “drug” is a substance that is pharmaceutically active, and a “pharmaceutically active ingredient” (API) is the pharmaceutically active substance in a drug.

The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent agent or compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, e.g., Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977). In some embodiments, a “pharmaceutically acceptable salt” is intended to mean a salt of a free acid or base of an agent or compound represented herein that is non-toxic, biologically tolerable, or otherwise biologically suitable for administration to the subject (see, generally, Berge, et al., J. Pharm. Sci., 1977, 66, 1-19). Preferred pharmaceutically acceptable salts are those that are pharmacologically effective and suitable for contact with the tissues of subjects without undue toxicity, irritation, or allergic response. An agent or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosul fates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, [gamma]-hydroxybutyrates, glycolates, tartrates, and mandelates.

Sodium azide (NaN₃) is a highly toxic chemical. Due to its widespread production and ease of accessibility^{1,9}, sodium azide is a highly hazardous chemical and is listed as a Chemical of Interest by the Department of Homeland Security because it can cause mass casualties if high amounts were released in an industrial accident or terrorist attack¹⁴. Azide-poisoned patients can present with vomiting, seizures, hypotension, metabolic acidosis, and coma and/or death can occur. No specific azide antidote exists currently, with treatment being solely supportive. Although poisoning is rare, the infrequency of reported cases means most medical personnel will not possess sufficient knowledge to properly treat azide-poisoned victims. This poor knowledge in conjunction with the absence of an azide-specific antidote may have contributed to varied success in treating patients. Thus, an urgent need exists for an azide antidote that can be administered easily, especially in a mass-casualty event.

The mechanism of sodium azide-induced toxicity is likely due to the combination of cytochrome c oxidase inhibition and increased nitric oxide production²¹⁻²³. Azide inhibits mitochondrial cytochrome c oxidase and is likely oxidized to nitric oxide (NO) in vivo. Cytochrome c oxidase inhibition depletes intracellular ATP and increases oxidative stress, while increased NO can cause hypotension and exacerbate oxidative damage. Sodium azide was therefore found to decrease cell growth and intracellular ATP content, and increase apoptosis and oxidative stress in mammalian cells. The decrease in ATP and increase in apoptosis and oxidative stress were likely from cytochrome c oxidase inhibition, with an attendant increase in electron leakage from the mitochondrial electron transport chain. Electron leakage decreases the mitochondrial membrane potential, thereby decreasing ATP synthase activity and increasing O₂⁻ and hydrogen peroxide production. These reactive oxygen species lead to apoptosis through mitochondrial release of cytochrome c and apoptosis-inducing factor³⁹, and to oxidative stress by oxidizing DNA, lipids, and proteins. Thus, within cultured cells, all of the toxicity of azide could be explained by cytochrome c oxidase inhibition. However, this seems not to be the case in mice, where the observed hypotension was more likely from increased NO production as assessed by a marked increase in serum nitrite and nitrate concentrations. Both cytochrome c oxidase inhibition and increased NO production are important mechanisms for azide toxicity.

Cobinamide is well suited as an azide antidote for the following three reasons. First, cobinamide is a potent and versatile antioxidant, serving as both a superoxide dismutase mimetic and a catalase mimetic, thereby neutralizing O₂⁻ and hydrogen peroxide, respectively³⁴. Cobinamide also neutralizes peroxynitrite, a strong oxidizing species that is generated by the diffusion-limited reaction of O₂⁻ and NO³⁴. Second, cobinamide oxidizes NO to nitrite and then the resulting reduced form of cobinamide binds NO: thus, one cobinamide molecule can neutralize two NO molecules³⁰. And third, cobinamide binds azide, and thus could potentially neutralize azide directly. However, the affinity of cobinamide for azide is relatively modest, and thus this mechanism would likely play a role only when azide is present at relatively high concentrations.

Although no drug is approved for azide poisoning, cobalamin has been used and it is a reasonable treatment due to its binding of azide and its antioxidative properties^{26,31,34}. However, cobinamide is superior to cobalamin in rescuing cells, fruit flies, and mice from azide exposure. Thus, cobinamide is favorable to cobalamin for treating azide poisoning.

While cobinamide bound azide with a moderate affinity (Kₐ 2.87×10⁵ M⁻¹), cobinamide improved growth, increased intracellular ATP, and reduced apoptosis and malondialdehyde, a marker of oxidative stress, in azide-exposed cells. Cobinamide also rescued D. melanogaster and mice from lethal exposures to azide and was more effective than cobalamin. Azide likely generated NO in the mice, as evidenced by increased serum nitrite and nitrate and reduced blood pressure and peripheral body temperature in the animals; the reduced temperature was likely due to reflex vasoconstriction in response to the hypotension. Cobinamide improved recovery of both blood pressure and body temperature.

Although the rate of azide conversion to NO in an animal is unknown, it likely is fast since the observed blood pressure in mice dropped by ~50% within 5 minutes of azide exposure. Consistent with a swift in vivo conversion of azide to NO, serum concentration of nitrite and nitrate was found to increase rapidly in the mice. Within 15 minutes of an intramuscular injection of cobinamide, the blood pressure of the mice began to rise, likely due to cobinamide's neutralization of NO.

Other agents have shown some benefit as azide antidotes, including the azide scavenger Co(II)N₄[11.3.1]^{23,41} and the antioxidant quercetin²⁰. Thus, scavenging azide and neutralizing reactive oxygen species are reasonable approaches for antagonizing azide's toxic effects. However, cobinamide could prove superior to these other agents due to cobinamide's multiple effects, including neutralizing nitric oxide. That is, because cobinamide neutralizes both oxidative stress and NO, it should be given further consideration as a sodium azide antidote.

In an aspect, a method of treating azide poisoning in a subject is provided, where the method includes administering to the subject an effective amount of cobinamide, a cobinamide derivative, or a salt thereof. In embodiments, the subject is a human, and the effective amount is from about 20 mg/kg to about 40 mg/kg, preferably about 30 mg/kg. In some embodiments, the cobinamide, cobinamide derivative, or salt thereof is administered orally. In other embodiments, the cobinamide, cobinamide derivative, or salt thereof is administered by intravenous injection. In embodiments, the azide poisoning is due to sodium azide exposure.

In embodiments, the azide poisoning is treated by increasing intracellular adenosine triphosphate (ATP) in azide-exposed cells. In embodiments, the azide poisoning is treated by inhibiting an increase in malondialdehye in azide-exposed cells. In embodiments, the azide poisoning is treated by decreasing apoptosis in azide-exposed cells.

In another aspect, a pharmaceutical composition is provided, where the pharmaceutical composition includes an effective amount of cobinamide, a cobinamide derivative, or a salt thereof, and where the pharmaceutical composition is effective to treat azide poisoning in a human subject. In embodiments, the azide poisoning is due to sodium azide exposure. In embodiments, the effective amount is from about 20 mg/kg to about 40 mg/kg, preferably about 30 mg/kg. In some embodiments, the pharmaceutical composition is configured for oral administration. In other embodiments, the pharmaceutical composition is configured for intravenous administration.

In embodiments, the pharmaceutical composition is effective to increase intracellular adenosine triphosphate (ATP), inhibit an increase in malondialdehye, and/or decrease apoptosis in azide-exposed cells. In embodiments, the pharmaceutical composition is effective to increase blood pressure and/or peripheral blood pressure in response to azide poisoning-induced vasoconstriction and/or hypotension.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1. Materials and Methods

### Materials

Aquohydroxocobinamide was synthesized by base hydrolysis of cobalamin (Nutrakey Industries) with purification over reversed-phase resins¹⁶. Histidyl-cobinamide was generated by adding two molar equivalents of histidine (Sigma-Aldrich, St. Louis, MO) to an aqueous solution of aquohydroxocobinamide. The histidyl ligand was added to increase cobinamide absorption after intramuscular injection. The term “cobinamide” is used generically in the text without reference to the axial ligands. All experiments were performed with cobinamide in the +3 oxidation state.

### Assessment of Azide Binding to Cobinamide and Cobalamin

Binding of ligands to the cobalt center of cobinamide or cobalamin leads to spectral changes in the UV-visible range. The ultra-violet-visible spectra of aquohydroxocobinamide and hydroxocobalamin were recorded on a Kontron 960 double-beam spectrophotometer in the absence and presence of varying concentrations of sodium azide. Spectra were recorded after incubating 25 μM aquohydroxocobinamide or 25 μM hydroxocobalamin with indicated amounts of sodium azide for 5 min at room temperature in 20 mM sodium phosphate buffer, pH 7.4.

### Tissue Culture

A549 type II human alveolar adenocarcinoma cells and COS-7 monkey kidney cells were obtained from the American Type Culture Collection. They were grown in Dulbecco's Modified Eagles Medium (DMEM) supplemented with 25 mM glucose and 10% fetal bovine serum (FBS).

### Cell Survival Studies

Approximately 5×10⁴ A549 or COS-7 cells were seeded in six-well plates overnight. Cells were then treated with sodium azide at the indicated concentrations for 48 h in the presence or absence of 25 μM aquohydroxocobinamide or 25 μM hydroxocobalamin. After treatment, cells were washed with phosphate-buffered saline (PBS) and counted using a hemocytometer.

To assess clonogenic survival, A549 cells were seeded overnight at a low density (~100 cells per six-well plate). The cells were then exposed for 24 hours with sodium azide at the indicated concentrations in the presence or absence of 25 μM aquohydroxocobinamide. The cells were washed with PBS and incubated in drug-free medium for 15 days. To evaluate colony number, cells were first washed in PBS and then stained with 0.25% w/v crystal violet in 25% methanol overnight. Following removal of the stain, the plates were air-dried and colonies were counted by visual inspection.

### Measuring Intracellular ATP Content

A549 and COS-7 cells were seeded in 24-well plates in standard growth medium and then incubated for 24 hours in glucose-free medium supplemented with 25 mM galactose (as a carbon source) and 10% PBS. The cells were then treated for 24 hours with 0.1 mM NaN₃ in the presence or absence of 25 μM aquohydroxocobinamide. Adenosine triphosphate (ATP) was measured using the Cell-Titer Glo 2.0 kit (Promega, Madison, WI). Briefly, cells were lysed in situ and ATP was measured in the lysates using the firefly luciferase-luciferin system. Luminescence was recorded using a Tecan Infinite™ 200 Microplate Reader. The amount of ATP was normalized to 100,000 cells.

### Measuring Lipid Peroxidation Products

A549 cells in 10-cm plates were treated with indicated amount of azide with or without 25 μM aquohydroxocobinamide for 24 hours. The cells were harvested using trypsin/EDTA, counted with a hemocytometer, and washed with PBS. The cells were lysed in H2O and extracted in 3% trichloroacetic acid/33% thiobarbituric acid. The extracts were boiled for 45 minutes and lipids were extracted in butanol. Malondialdehyde, a product of lipid peroxidation, reacts with thiobarbituric acid to generate a pink-colored species that was measured spectrofluorometrically at 530ex and 590em. The amount of malondialdehyde in samples was determined from standard curves (Cayman Chemical Company, Ann Arbor, MI), and recovery of malondialdehyde in spiked samples was greater than 90%.

### Detecting Cleaved Caspase-3

A549 cells were plated on glass coverslips and then treated with indicated amounts of azide in the presence or absence of 25 μM cobinamide for 24 hours. The cells were fixed in 3.7% paraformaldehyde, permeabilized with 1% Triton-X-100, and incubated with a cleaved caspase-3-specific antibody (1:100 dilution) (Cell Signaling, Danvers, MA) overnight at 4° C. After incubation with a FITC-conjugated secondary antibody (1:100 dilution) (Jackson ImmunoResearch, West Grove, PA), nuclei were stained using Hoechst 33342 dye. Immunofluorescence images were recorded using a Keyence BZ-X700 fluorescence microscope.

### Drosophila Melanogaster Poisoning Models

Drosophila melanogaster were raised on 400 μM aquohydroxocobinamide or 400 μM hydroxocobalamin for at least one week to preload the animals with the drugs. Animals of both sexes and various ages were included to represent the heterogeneity of the general population that would be exposed to azide in an industrial accident or terrorist attack.

To simulate azide inhalation, hydrazoic acid (HN₃), which is volatile at room temperature, was generated by adding 100 μL of a 100 mM sodium azide solution to a 1 cm² piece of Whatman paper that had been previously saturated with 1 N hydrochloric acid and then air-dried. The Whatman paper was immediately transferred to vials containing D. melanogaster and the number of flies falling motionless over 2 minutes was recorded. The flies were then transferred to new vials and were monitored for recovery over 3 days.

To simulate azide ingestion, gauze soaked in 100 mM sodium azide or sodium phosphate was placed into empty vials, with the gauze as the only source of water. Flies were transferred to the vials and their survival was recorded over 8 hours.

### Mouse Poisoning Models

Male and female C57BL/6J mice aged 3-21 months were purchased from Jackson Laboratory (Bay Harbor, ME); using both sexes and varying-aged mice simulated a heterogenous human population, as would be present in a mass casualty event. The experiments were conducted according to the National Academies of Sciences, Engineering, and Medicine Institute for Laboratory Animal Research Guide to the Care and Use of Laboratory Animals and were approved by the Institutional Animal Care and Use Committee (IACUC) at the University of California, San Diego.

In survival studies, mice were anesthetized with 2% isoflurane and then given 30 mg/kg sodium azide by intraperitoneal injection as a 100-μL bolus from a 1 ml plastic syringe. This dose was empirically determined to be ≥75% lethal by exposing four groups of mice, 4-6 animals per group, to varying amounts of sodium azide ranging from 25 mg/kg to 33 mg/kg. Two minutes after the sodium azide injection, the mice were given an intramuscular injection of 350 mg/kg histidylcobinamide, 450 mg/kg hydroxocobalamin, or water vehicle into the hind quadricep. The animals were removed from isoflurane and monitored for survival over 3 hours. Survivors were returned to the vivarium. The cobinamide dose is the same as that used to rescue mice from hydrogen sulfide poisoning¹⁸, and corresponds to 28.4 mg/kg when converted to a human equivalent dose using body surface area correction; the cobalamin dose corresponds to the cobinamide dose on a molar basis.

To evaluate the effect of sodium azide on blood pressure, the systolic and diastolic blood pressure was measured by tail-cuff plethysmography (Kent Scientific Corporation CODA system, Torrington, CT). The occlusion cuff was placed 10 mm from the base of the tail and the volume pressure recording cuff was placed 10 mm distal to the occlusion cuff. Mice were then given an intraperitoneal injection of water or 20 mg/kg sodium azide as a 100-μL bolus. Blood pressure was measured every 5 minutes for 20 minutes, at which time the mice were given one intraperitoneal injection of either 350 mg/kg histidylcobinamide or water vehicle as a 100-μL bolus. The blood pressure was measured every 5 minutes over a 15-minute period and then at 60, 120, and 180 minutes after the sodium azide injection.

The same treatment protocol as described for measuring blood pressure was applied to measuring tail vein temperature. Measurements were taken at indicated times using a laser thermometer. To assure temperature was taken at the same location each time, a spot was marked 20 mm from the base of the tail. Room temperature was also recorded during the duration of the tail temperature assessment.

### Measurement of Serum Nitrite and Nitrate

Mice were given an intraperitoneal injection of 20 mg/kg sodium azide (or water vehicle) as a 100-μL bolus, and then euthanized via carbon dioxide asphyxiation at indicated times. Blood was collected immediately via cardiac puncture and centrifuged to remove cells. Nitrite and nitrate were measured in the serum using the Griess reagent-based assay. However, azide interferes with the assay³⁷; to circumvent this problem, azide was eliminated from samples by first converting it to hydrazoic acid, which was then removed by evaporation. Briefly, serum was deproteinated using centrifugal concentrators with a 5000 Dalton molecular weight cut-off. The samples were then acidified using hydrochloric acid (HCl), incubated for 2 hours at 37° C., and neutralized with sodium hydroxide (NaOH) and 50 mM Tris pH 7.4. Sample volumes were equalized, and the samples were incubated with nitrate reductase (Active Motif, Carlsbad, CA) to reduce nitrate to nitrite. Griess reagent (Sigma-Aldrich, St. Louis. MO) was then added to each sample, and after a 20-minute incubation period, absorbance was measured at 540 nm in a microplate reader. The amount of nitrite and nitrate in samples was determined from standard curves, and the recovery of nitrite and nitrate in spiked samples was greater than 90%.

### Statistics

Statistical tests were performed with GraphPad Prism Statistics Software Version 9.3.0 (GraphPad Prism, Carlsbad, CA). Data are presented as the mean (or the mean normalized to controls set at 100%)±SEM. Both one-way and two-way ANOVAs with Šadák correction were used to detect differences in cell-based assays. In the D. melanogaster studies and in the mouse studies where serum nitrite and nitrate concentrations were measured, blood pressure, and peripheral body temperature, differences among conditions were assessed using a two-way ANOVA with Šadák correction. In the mouse survival studies, a log-rank test was used to assess differences. A p-value<0.05 was considered significant.

### Example 2. Results

Cobinamide Binds Azide and Protects Cultured Mammalian Cells from Azide Toxicity

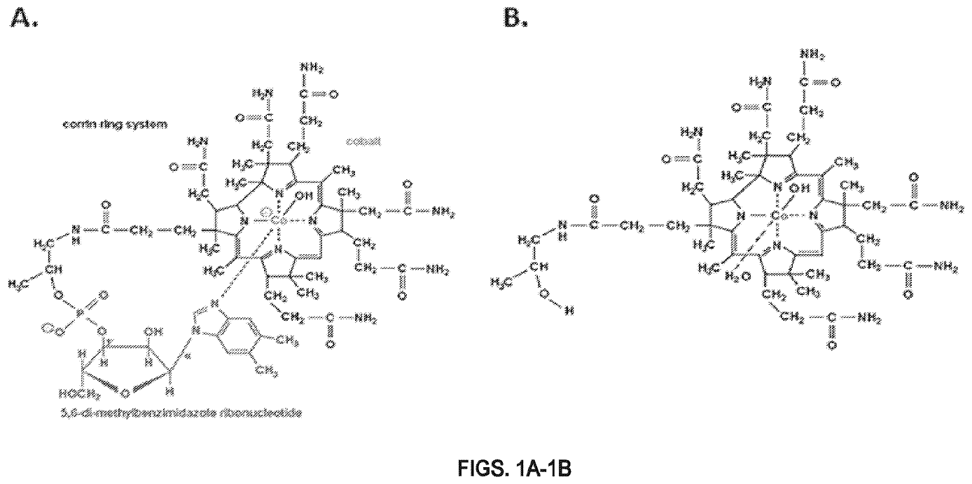

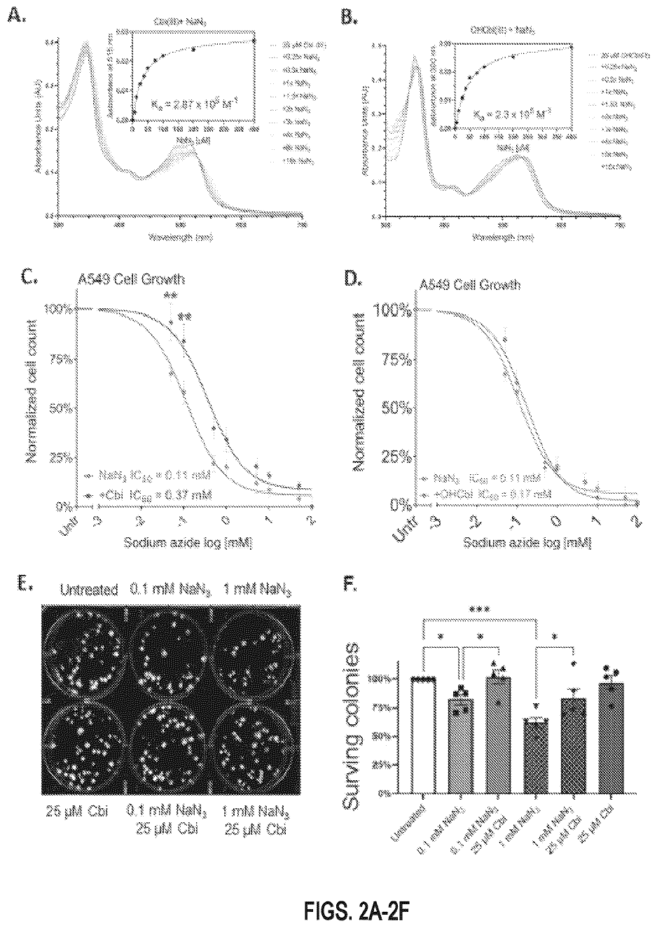

Since cobalamin binds azide³¹, it was hypothesized that cobinamide would also bind azide. Increasing concentrations of sodium azide were added, from 6.25 to 400 μM, to a 25 μM aquohydroxocobinamide solution and found a progressive change in the UV-visible spectrum of cobinamide (FIG. 2A). From these data, it was estimated that a Kₐ of azide binding to cobinamide of 2.87×10⁵ M⁻¹ based on the absorbance change at 515 nm (FIG. 1A). For comparison, we found a Kₐ of azide binding to hydroxocobalamin of 2.3×10⁵ M⁻¹, which is similar to the published value³¹ of 7.2×10⁴ M⁻¹ (FIG. 2B). Although cobinamide had a slightly higher affinity for azide than cobalamin, both binding affinities are rather modest.

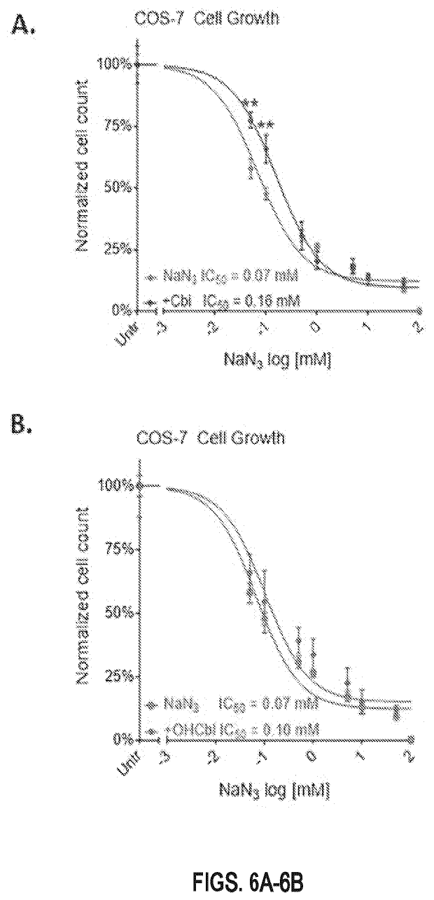

To determine if cobinamide reduced azide toxicity in cells, cobinamide was tested in COS-7 and A549 cells exposed to sodium azide. We found that 25 μM aquohydroxocobinamide increased the IC₅₀ of sodium azide by about 3.4-fold in A549 cells (FIG. 2C), but 25 μM hydroxocobalamin had essentially no effect on reducing sodium azide toxicity in these cells (FIG. 2D). A549 cells were derived from a patient with a lung adenocarcinoma and can generate clones from single cells, allowing analysis of the effect of sodium azide on clonal growth. It was found that 0.1 mM and 1 mM sodium azide significantly reduced A549 cell clonal growth, and that 25 μM aquohydroxocobinamide improved growth at both azide concentrations (FIGS. 2E-2F). Similar to A549 cells, 25 μM aquohydroxocobinamide increased the IC₅₀ of sodium azide by about 2.3-fold in COS-7 cells, but 25 μM hydroxocobalamin led to only a marginal reduction in azide toxicity (FIGS. 6A-6B). Since cobinamide and cobalamin had a similar Kₐ for azide and yet only cobinamide reduced azide toxicity, this suggests cobinamide was ameliorating azide toxicity through mechanism(s) other than binding azide.

### Mechanisms of Azide Toxicity, Rescue by Cobinamide

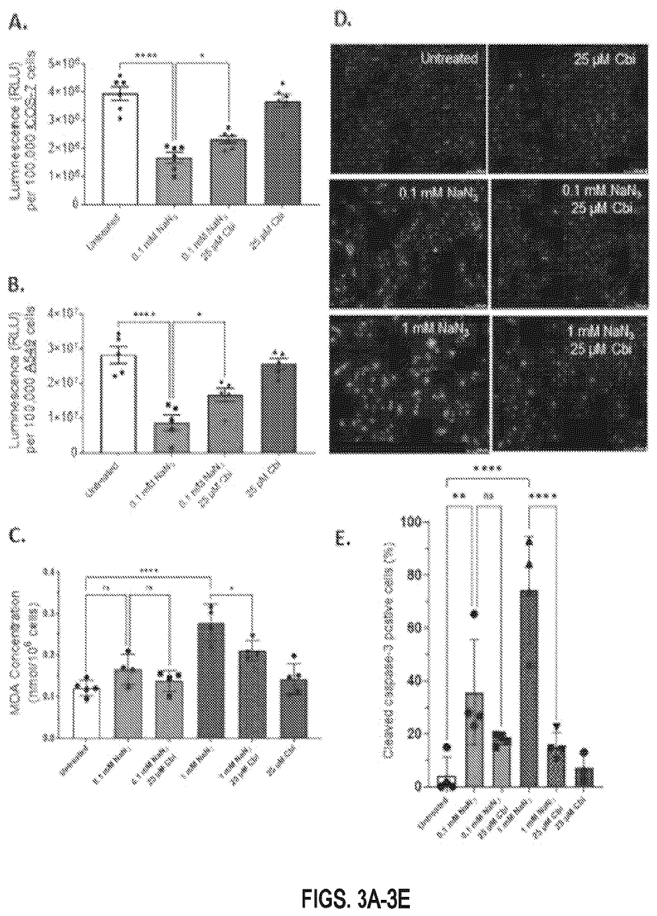

The effect of azide on cellular metabolites has received little attention. Since azide inhibits mitochondrial cytochrome c oxidase, it was predicted that azide would reduce the intracellular concentration of ATP. Exposing COS-7 and A549 cells for 24 hours to 0.1 mM sodium azide (near the IC₅₀ for both cell types) was found to markedly decrease the amount of intracellular ATP in both cell types (FIGS. 3A-3B). Cobinamide significantly increased intracellular ATP in the azide-treated cells (FIGS. 3A-3B).

Inhibition of cytochrome c oxidase increases electron leakage from the electron transport chain, and this can result in increased generation of superoxide and increased oxidative stress²⁸. Intracellular malondialdehyde was assessed as a readout for lipid oxidation, and it was found that while 0.1 mM sodium azide marginally increased malondialdehyde in A549 cells, 1 mM sodium azide significantly increased the malondialdehyde concentration (FIG. 3C). Consistent with cobinamide being a strong antioxidant³⁴, it was found that it significantly reduced the increase in malondialdehyde by 1 mM sodium azide (FIG. 3C).

The combination of ATP depletion and increased oxidative stress could increase cellular apoptosis. It was found that azide caused a dose-dependent increase in apoptosis as assessed by measuring cleaved caspase-3 positive cells (FIGS. 3D-3E). Cobinamide significantly reversed apoptosis induced by 1 mM sodium azide.

Cobinamide Rescues Flies and Mice from Sodium Azide Toxicity

Whether cobinamide could rescue an intact animal from azide toxicity was also tested. In the initial studies, D. melanogaster were used, which are increasingly used in drug discovery due to their low cost, rapid life cycle, and wide availability of genetic variants. Two common modes of azide poisoning were tested: inhalation and ingestion¹⁴. In both models, aquohydroxocobinamide or hydroxocobalamin was pre-administered to the flies for one week by adding the drugs to their food.

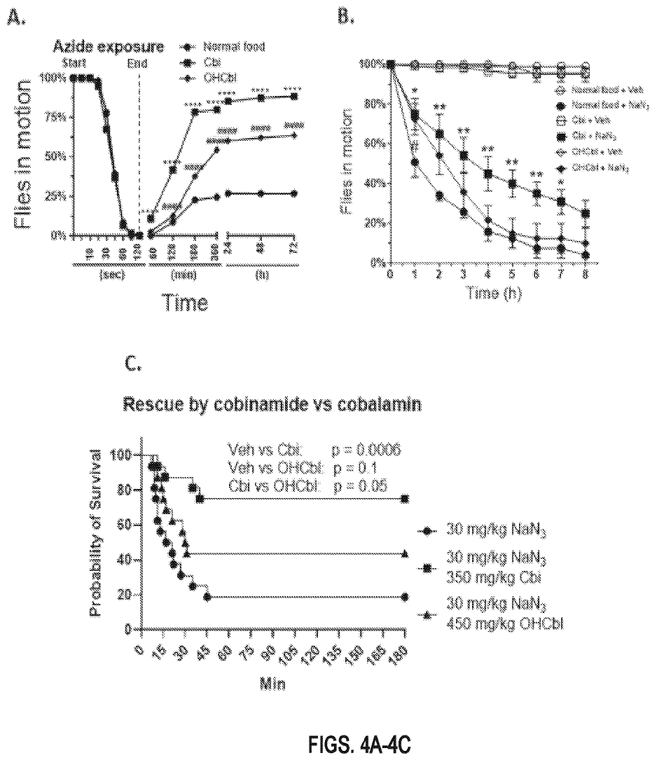

To simulate inhalation toxicity, the flies were exposed to hydrazoic acid (HN₃), which was generated in a tightly-capped vial by spotting NaN₃ on acidified filter paper. The calculated HN₃ concentration in the vial was 1500 ppm. All flies became motionless within 2 minutes following HN₃ exposure, regardless of whether they had been pre-treated with cobinamide or cobalamin (FIG. 4A). This rapid state of immobility recapitulated the “knock-down” and unconscious state experienced by humans exposed to concentrated hydrazoic acid³⁸. The flies were then moved to vials containing standard food and monitored over 3 days. Flies raised on both aquohydroxocobinamide and hydroxocobalamin showed better recovery than control animals, with cobinamide being superior to cobalamin (FIG. 4A).

To simulate oral poisoning, which is the main route of azide exposure in humans¹⁴, flies were placed in vials containing a gauze soaked in 100 mM NaN₃ or sodium phosphate as their only source of water. The flies were monitored for survival over 8 hours. Flies that had received hydroxocobalamin showed a marginal improvement in survival, whereas flies that had received aquohydroxocobinamide showed a major improvement in survival (FIG. 4B).

It was then tested whether cobinamide could improve survival in mice that received a lethal dose of sodium azide administered by intraperitoneal injection. Aquohydroxocobinamide or hydroxocobalamin were administered by intramuscular injection, the preferred route for administering drug in a pre-hospital setting, as in a large casualty event. It was found that cobinamide-treated mice, both males and females, showed significantly better survival than their control counterparts (FIG. 4C). Although cobalamin-treated mice showed some improvement in survival compared to control mice, the difference between the two groups was not significant and rescue by cobinamide was significantly different than that by cobalamin.

### Therapeutic Effect of Cobinamide on Blood Pressure and Peripheral Body Temperature

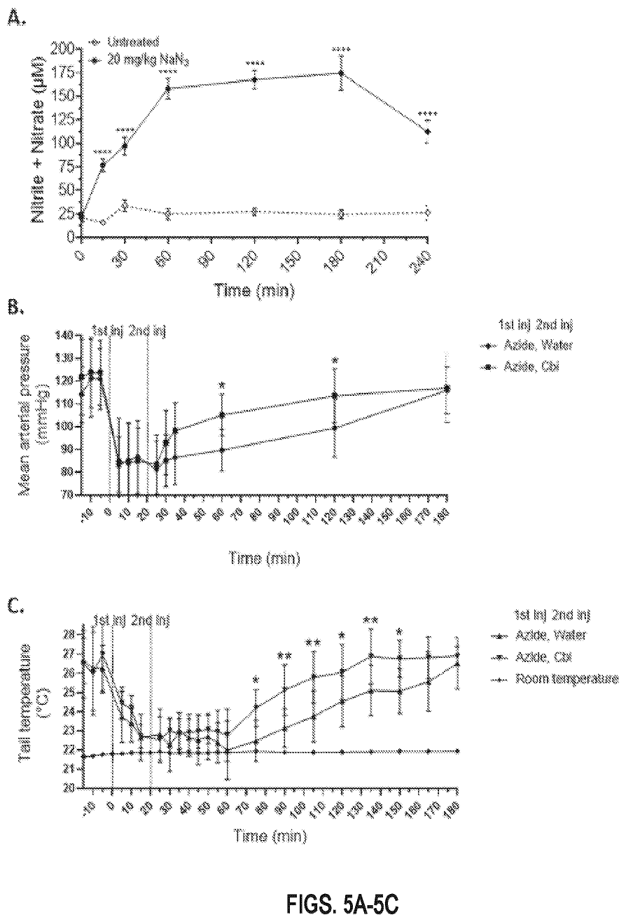

Sodium azide has been shown to increase nitric oxide generation in vivo through an undefined mechanism²¹⁻²³. Nitric oxide is rapidly oxidized to nitrite and nitrate and the latter can be used as a proxy for nitric oxide concentration. It was found that serum nitrite and nitrate concentrations rose rapidly and remained elevated for 3 hours following sodium azide injection into both male and female mice (FIG. 5A). Serum nitrite and nitrate concentrations could not be measured in mice receiving cobinamide.

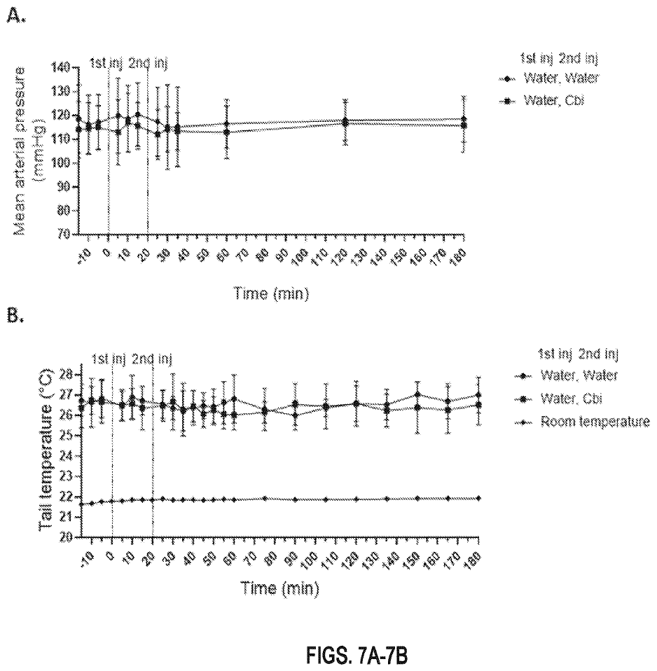

Since nitric oxide is a potent vasodilator, changes in blood pressure were assessed in mice following a sub-lethal dose of azide. Within 5 minutes of receiving an intraperitoneal injection of sodium azide, the mean arterial pressure (MAP) decreased by 30-40 mm Hg (FIG. 5B). At 20 minutes after azide exposure, comparable to the amount of time required for emergency medical personnel to reach the scene of a poisoning event, mice received either water or 300 mg/kg histidylcobinamide by intraperitoneal injection. Although blood pressure began to rise in both conditions, recovery was more rapid among animals that received cobinamide compared to those that received vehicle (water). Mice given vehicle or cobinamide alone had a steady MAP throughout the study (FIG. 7A).

Peripheral vasoconstriction is a compensatory mechanism to increase blood flow to visceral organs and the brain under conditions of hypotension: this can lead to peripheral hypothermia. In alignment with the decreased blood pressure after sodium azide injection, tail temperature dropped, albeit not as rapidly as the fall in blood pressure (FIG. 5C). In contrast to the MAP which reached its nadir within 5 minutes post azide exposure, tail temperature reached its nadir 20 minutes post-exposure. Room temperature remained steady throughout the experiment. Following injection of either the water vehicle or cobinamide, the tail temperature remained low for ~40 minutes, and then increased at a significantly faster rate in cobinamide- than vehicle-treated animals. Treatment with water or cobinamide alone did not affect tail temperature (FIG. 7B).

### REFERENCES

1. McKeen S. High Production Volume (HPV) Chemicals. Paris (France): The Organization for Economic Co-Operation and Development; Environment Directorate. 2010.
2. Chang S, Lamm S H. Human health effects of sodium azide exposure: a literature review and analysis. International Journal of Toxicology. 2003 May-June; 22(3):175-86.
3. Tat J. Heskett K, Satomi S, et al. Sodium azide poisoning: a narrative review. Clinical Toxicology. 2021:1-15.
4. The Japan Times. Poisonings uncover lax sodium azide controls. The Japan Times. 1998 Oct. 30 Sect. National.
5. Zambito T. Man admits making deadly toxins Jersey City ex-pharmacist also stockpiled cache of weapons. The Star-Ledger (Newark, NJ). 2014 2014 May 30 [cited 2019 Nov. 13].
6. Agence France-Presse. Czech embassy in Slovakia gets poisoned mail. Agence France-Presse. 2014 2014 Dec. 12 [cited 2019 Nov. 12].
7. Pankratz H. Raided building evacuated again Chemist may have poured compounds in sink. The Denver Post, (Denver, CO). 2002 2002 Aug. 16 [cited 2019 Nov. 13]; B-02.
8. US Fed News (USA). Pipe bomb crafted from vehicle airbag gets birmingham man 10 years in federal prison. US Fed News (USA). 2014 2014 Aug. 28 [cited 2019 Nov. 13].
9. Leonard J B, Hines E Q, Anderson B D. Prime eligible poisons: identification of extremely hazardous substances available on Amazon.com. Clinical Toxicology. 2020; 58(1):45-48.
10. The Times Argus. Harvard: Lab workers poisoned by tainted coffee. Times Argus, The (Montpelier-Barre, VT). 2009 2009 Oct. 26 [cited 2019 Nov. 13].
11. New Haven Register. Sodium azide may have caused illness at Yale School of Medicine in New Haven, officials say. New Haven Register (CT). 2017 2017 Mar. 8 [cited 2019 Nov. 12].
12. Hirose Y, Hata K. Honda H, et al. Clinical study of a sodium azide poisoning cluster. Nihon Kyukyu Igakukai Zasshi. 2001; 12(3):125-129.
13. Schwarz E S, Wax P M, Kleinschmidt K C, et al. Multiple poisonings with sodium azide at a local restaurant. The Journal of Emergency Medicine. 2014; 46(4):491-494.
14. Holstege C P, Bechtel L K. Reilly T H. et al. Unusual but potential agents of terrorists. Emergency Medicine Clinics of North America. 2007; 25(2):549-566.
15. Workum J D, Bisschops L L, van den Berg M J. Autointoxication with ‘suicide powder’. Nederlands Tijdschrift Voor Geneeskunde. 2019; 163.
16. Keilin D, Hartree E. Inhibitors of catalase reaction. Nature. 1934; 134(3398):933.
17. Stannard J, Horecker B. The in vitro inhibition of cytochrome oxidase by azide and cyanide. Journal of Biological Chemistry. 1948; 172(2):599-608.
18. Jiang J, Chan A, Ali S, et al. Hydrogen Sulfide—Mechanisms of Toxicity and Development of an Antidote. Sci Rep. 2016 Feb. 15; 6:20831.
19. Philipopoulos G P. Tat J, Chan A, et al. Methyl mercaptan gas: mechanisms of toxicity and demonstration of the effectiveness of cobinamide as an antidote in mice and rabbits. Clin Toxicol (Phila). 2022 May; 60(5):615-622.
20. Somade O T, Olorode S K, Olaniyan T O, et al. Quercetin, a polyphenolic phytochemical prevents sodium azide-induced extra-hepatic oxidative stress in rats. Cogent Biology. 2016; 2(1):1200798.
21. Kruszyna H, Kruszyna R, Smith R P, et al. Red blood cells generate nitric oxide from directly acting, nitrogenous vasodilators. Toxicology and Applied Pharmacology 1987:91(3):429-438.
22. Kruszvna R. Kruszvna H. Smith R P, et al. Generation of valency hybrids and nitrosylated species of hemoglobin in mice by nitric oxide vasodilators. Toxicology and Applied Pharmacology 1988; 94(3):458-465.
23. Frawley K L, Carpenter Totoni S, Bae Y, et al. A comparison of potential azide antidotes in a mouse model. Chemical Research in Toxicology. 2020; 33(2):594-603.
24. Lopacinski B, Kolacinski Z, Winnicka R. Sodium azide—clinical course of the poisoning and treatment. Przeglad Lekarski. 2007; 64(4-5):326-330.
25. Meatherall R, Palatnick W. Convenient headspace gas chromatographic determination of azide in blood and plasma. Journal of Analytical Toxicology. 2009; 33(8):525-531.
26. Bartecka-Mino K, Schiel H, Holzer A, et al., editors. Hydroxocobalamin: An antidote for sodium azide poisoning? Clinical Toxicology; 2014: Informa Healthcare 52 Vanderbilt Ave, New York, NY 10017 USA.
27. Anusiem A, Beetlestone J, Irvine D. Reactivity differences between haemoglobins. Part VIII. The thermodynamics of the reaction of human methaemoglobins A and C with fluoride, thiocyanate, and cyanide ions. An interpretation of enthalpy changes in terms of hydration. Joumal of the Chemical Society A: Inorganic, Physical, Theoretical. 1968:960-969.
28. Abbanat R A, Smith R P. The influence of methemoglobinemia on the lethality of some toxic anions: I. Azide. Toxicology and Applied Pharmacology 1964; 6(5):576-583.
29. Rochelle L G, Morana S J, Kruszyna H, et al. Interactions between hydroxocobalamin and nitric oxide (NO): evidence for a redox reaction between NO and reduced cobalamin and reversible NO binding to oxidized cobalamin. Journal of Pharmacology and Experimental Therapeutics 1995; 275(1):48-52.
30. Sharma V S, Pilz R B, Boss G R, et al. Reactions of nitric oxide with vitamin B12 and its precursor, cobinamide. Biochemistry. 2003:42(29):8900-8908.
31. Firth R, Hill H, Pratt J, et al. The chemistry of vitamin B 12. Part XI. Some further formation constants. Journal of the Chemical Society A: Inorganic, Physical, Theoretical. 1969:381-386.
32. Broderick K E, Potluri P, Zhuang S, et al. Cyanide detoxification by the cobalamin precursor cobinamide. Experimental Biology & Medicine. 2006:231(5):641-649.
33. Hayxward G, Hill H, Pratt J, et al. 1196. The chemistry of vitamin B 12. Part IV. The thermodynamic trans-effect. Journal of the Chemical Society. 1965:6485-6493.
34. Chang S, Tat J, China S P, et al. Cobinamide is a strong and versatile antioxidant that overcomes oxidative stress in cells, flies, and diabetic mice. PNAS Nexus. 2022.
35. Broderick K E, Singh V, Zhuang S, et al. Nitric oxide scavenging by the cobalamin precursor cobinamide. Journal of Biological Chemistry. 2005 Mar. 11:280(10):8678-85.
36. Salnikov D S, Makarov S V, van Eldik R, et al. Kinetics and mechanism of the reaction of hydrogen sulfide with diaquacobinamide in aqueous solution. European Journal of Inorganic Chemistry. 2014; 2014(25):4123.
37. Nims R W, Cook J C, Krishna M C, et al. Colorimetric assays for nitric oxide and nitrogen oxide species formed from nitric oxide stock solutions and donor compounds. Methods in Enzymology. Vol. 268: Elsevier; 1996. p. 93-105.
38. Dyer J E. Azide, sodium. In: Olson K R, editor. Poisoning & Drug Overdose. 6 ed: The McGraw-Hill Companies; 2012.
39. Redza-Dutordoir M, Averill-Bates D A. Activation of apoptosis signalling pathways by reactive oxygen species. Biochimica et Biophysica Acta. 2016 December; 1863(12):2977-2992.
40. Praekunatham H, Garrett K K, Bae Y, et al. A Cobalt Schiff-Base Complex as a Putative Therapeutic for Azide Poisoning. Chemical Research in Toxicology. 2019; 33(2):333-42.
41. Frawley K L, Carpenter Totoni S, Bae Y, et al. A comparison of potential azide antidotes in a mouse model. Chem Res Toxicol. 2020 Feb. 17; 33(2):594-603. doi: 10.1021/acs.chemrestox.9b00422. PubMed PMID: 31922405.
42. Schwinn D A, McIntyre R W, Reves J G. Isoflurane-induced vasodilation: role of the alpha-adrenergic nervous system. Anesth Analg. 1990 November; 71(5):451-9.
43. Constantinides C, Mean R, Janssen B J. Effects of isoflurane anesthesia on the cardiovascular function of the C57BL/6 mouse. ILAR J. 2011:52(3):e21-31.

What is claimed is:

1. A method of treating azide poisoning in a subject comprising administering to the subject an effective amount of cobinamide, a cobinamide derivative, or a salt thereof.

2. The method of claim 1, wherein the subject is a human.

3. The method of claim 2, wherein the effective amount is from about 20 mg/kg to about 40 mg/kg, preferably about 30 mg/kg.

4. The method of claim 1, wherein the cobinamide, cobinamide derivative, or salt thereof is administered orally.

5. The method of claim 1, wherein the cobinamide, cobinamide derivative, or salt thereof is administered by intravenous injection.

6. The method of claim 1, wherein the azide poisoning is due to sodium azide exposure.

7. The method of claim 1, wherein azide poisoning is treated by increasing intracellular adenosine triphosphate (ATP) in azide-exposed cells.

8. The method of claim 1, wherein azide poisoning is treated by inhibiting an increase in malondialdehye in azide-exposed cells.

9. The method of claim 1, wherein azide poisoning is treated by decreasing apoptosis in azide-exposed cells.

10. A pharmaceutical composition comprising an effective amount of cobinamide, a cobinamide derivative, or a salt thereof, wherein the pharmaceutical composition is effective to treat azide poisoning in a human subject.

11. The pharmaceutical composition of claim 10, wherein the azide poisoning is due to sodium azide exposure.

12. The pharmaceutical composition of claim 10, wherein the effective amount is from about 20 mg/kg to about 40 mg/kg, preferably about 30 mg/kg.

13. The pharmaceutical composition of claim 10, wherein the pharmaceutical composition is configured for oral administration.

14. The pharmaceutical composition of claim 10, wherein the pharmaceutical composition is configured for intravenous administration.

15. The pharmaceutical composition of claim 10, wherein the pharmaceutical composition is effective to increase intracellular adenosine triphosphate (ATP), inhibit an increase in malondialdehyde, and/or decrease apoptosis in azide-exposed cells.

16. The pharmaceutical composition of claim 10, wherein the pharmaceutical composition is effective to increase blood pressure and/or peripheral blood pressure in response to azide poisoning-induced vasoconstriction and/or hypotension.
